(19) United States
(12) Patent Application Publication
(10) Pub. No.: US 2008/0095716 A1
(43) Pub. Date: Apr. 24, 2008

(54) Use in the Medical, Diagnostic and Phlebological Field of a Mixture of Sterile and Physiologically Acceptable Purposes

(75) Inventors: Lorenzo Tessari, Verona (IT); Attilio Cavezzi, Ascoli Piceno (IT); Antonio Luis Cabrera Garrido, Granada (ES)

Correspondence Address: James V Costigan;Hedman & Costigan, 1185 Avenue of the Americas, New York, NY, 10036-2646, US

(73) Assignee: KONNKLIJKE PHILIPS ELECTROINIC N.V., EINDHOVEN (NL)

(21) Appl. No.: 11/597,580

(22) PCT Filed: May 25, 2005
(86) PCT No.: PCT/IB05/01693

§ 371 Date: Jun. 19, 2007

(30) Foreign Application Priority Data
May 26, 2004 (IT) M12004A001056

(51) Int. Cl. A61B 8/00

(52) U.S. Cl. 424/9.52

(57) ABSTRACT

The present invention relates to a mixture of sterile and apyrogenic gases based on CO₂ and O₂ suitable for producing microbubbles which can be used in the medical, phlebological and diagnostic field. The gas mix allows stable and physiologically acceptable microbubbles to be formed, by means of more standardized procedures which are easy to effect.

The present invention relates to the use of a mixture of sterile and physiologically acceptable gases in the medical, phlebological and diagnostic field.

The present invention originates and can be applied in the diagnostic field, as a contrast means for echography, and in the medical field in phlebology and medical therapy in general.

In particular, the gas mix of the invention allows stable and physiologically acceptable sterile microbubbles to be produced, by means of a standardized and simple procedure.

The systemic use of contrast means for ultrasounds or ultrasoundgraphs (MCU) has been known for quite a time in the diagnostic field. A typical example of contrast means consists of microbubbles containing a bio-compatible gas. Once these microbubbles have been introduced into the human body, they cause a variation in the tissue characteristics of reflectivity, absorption and passage rate of the ultrasound beam. The main function of these contrast means is to increase the reflectivity of a tissue or organ with respect to the surrounding area.

This effect can be obtained by increasing the reflectivity, or rather the signal/sound ratio, thanks to the introduction of microbubbles into the bloodstream.

As microbubbles consisting of gases have an echo-genicity coefficient higher than the solid and liquid echografic means, when the surrounding medium is a liquid, as in the case of the bloodstream, the use of gases as a contrast medium in echography, echocardiography, vascular echocolordoppler, etc., has become increasingly more widespread.

When microbubbles based on gas are subjected to the action of ultrasounds, they cause a satisfactory refraction-reflection process so as to reveal, with echocolordoppler or echography, the relevant images as hyper-echogenic or chromatically typical.

Notoriously, the contrast means for ultrasounds based on gases must satisfy various conditions.

Firstly, they must be physiologically acceptable and have a sufficiently long persistence in the bloodstream to allow the necessary evaluations to be effected, for obtaining an adequate diagnostic response.

Furthermore, the microbubbles must have a sufficiently reduced dimension; this requirement jeopardizes the persistence, as it has been verified that once microbubbles having a diameter lower than 10 microns have been introduced into the circulatory flow, they only survive for 0.25 sec.

Gas having a high molecular weight was therefore used to produce bubbles, in an attempt to overcome this limit. The use of air and gases having a high molecular weight, such as C₂H₆, SF₆, C₄F₈ was also proposed for producing microbubbles.

It has been found however that the systemic administration of microbubbles containing said gases for echogenic purposes, can cause side-effects in the circulatory system, above all when the investigation is laborious and requires the administration of relevant amounts of gas.

Also in the phlebological field, where air has been widely used as a carrier medium of sclerosing substances, cases of transitory neurological deficits have been reported, as well as doubtful pulmonary embolisms; this makes it necessary to use reduced amounts of sclerosing foam and consequently of air.

In order to avoid the occurrence of these side-effects, resort was made to CO₂, a highly bio-compatible gas, as a carrier medium for sclerosing substances.

It was found however that although the use of CO₂ was able to cause a reduction in the appearance of side-effects in the circulatory system, it is not satisfactory with respect to the persistence of the microbubbles inside the syringe, before the injection of the sclerosing foam, and bloodstream. Once microbubbles containing carbon dioxide have been administered, they do in fact tend to explode or blend together forming bubbles with larger dimensions considered unsuitable for the purposes of sclerosing treatment. This is due to the greater diffusibility of CO₂ with respect to other gases.

The necessity is therefore currently felt for availing of methods for the production of biocompatible microbubbles which can be used in the medical, diagnostic and phlebological field.

One of the main objectives of the present invention consists in providing a mixture of physiologically acceptable gases, which can be used in a more or less standardized manner, for producing microbubbles suitable as carriers of sclerosing agents, or other drugs.

Another objective of the invention consists in providing a mixture of biocompatible sterile gases capable of forming microbubbles which are well tolerated and having persistence in the bloodstream suitable for effecting the evaluations necessary for obtaining an adequate diagnostic response.

A last objective is to provide a method for the extemporaneous formation of microbubbles whose systemic administration for diagnostic or therapeutic use is practically exempt from causing strong side-effects due to the gaseous component of the gas mix and active principle.

The applicants have found that it is possible to produce stabile microbubbles with safe medical use, by adopting a gaseous mixture based on two selected physiologically acceptable gases.

In view of the objectives indicated above and others which will appear more evident in the following description, a first aspect of the invention relates to the use of a composition of sterile gases, soluble at atmospheric pressure and physiologically acceptable for the production of microbubbles as a contrast means for ultrasounds, wherein said composition comprises oxygen and carbon dioxide and optionally at least another gas in a quantity not higher than 10% by volume with respect to the total volume of said mixture.

According to another aspect of the present invention, the use is provided of a composition of sterile gases, soluble at atmospheric pressure and physiologically acceptable for the production of microbubbles for sclerotherapy, wherein said composition comprises oxygen and carbon dioxide and optionally at least another gas in a quantity not higher than 10% by volume with respect to the total volume of said mixture.

The applicants have found that the use of oxygen and carbon dioxide, combined with each other, unexpectedly produce a synergic effect on the stabilization of the microbubbles formed, causing an increase in their persistence in the bloodstream which, in echographic diagnostics, allows the necessary evaluations to be effected for obtaining an adequate response and, in phlebological therapy, an adequate sclerosing treatment.

The mixture of O₂ and CO₂ is capable of effectively counterbalancing the surface tension of the microbubbles which is caused when they are inside the circulatory stream. Furthermore, the stabilization which is obtained with the combined use of CO₂ and O₂ is temporary, which allows an easy elimination of the microbubbles on the part of the organism thus avoiding any type of toxicity for the patient. When the microbubbles, in fact, collapse inside the blood veins, they release two non-toxic gases as they are physiologically already present in the organism.

In particular, the authors have found that a mixture of gases essentially based on O₂ and CO₂ has a higher density, a lower diffusibility and a lower saturation constant with respect to the air. The combination of these characteristics significantly contributes to optimizing the life duration of the microbubbles once they have been injected into the bloodstream.

Within the scope of the invention, the specific characteristics of CO₂ such as maximum tolerability, strong tendency to bind itself to the vein wall carrying the sclerosing drug, are synergically combined with those of oxygen obtaining a stabilization of the microbubbles which balances the greater tendency to coalescence that the same microbubbles would have if their single gas consisted of CO₂ alone.

Within the scope of the present invention, the term microbubbles is meant to comprise bubbles having a diameter ranging from 5 to 200 and preferably from 10 to 50 microns.

The formation of microbubbles with these dimensions minimizes the risks of embolism and the creation of side-effects which are dangerous for the patient's health.

In the mixture of the invention, the volume ratios between O₂ and CO₂ can typically vary within a wide range, for example from 5% to 95% by volume. It has been found, however, that when CO₂ is present within a range of 50 to 95% by volume and preferably 60-80% by volume, the mixture proves to be highly appropriate for the formation of particularly stable and biocompatible microbubbles.

Other gases are advantageously not present in the mixture of the invention, or should they be so, these additional gases are present in a quantity which does not exceed 10% by volume with respect to the final gaseous mix.

According to an embodiment, the composition of the invention can also comprise one or more biologically acceptable gases such as, for example, air, nitrogen, ozone. As previously specified, these gases, when present, do not exceed 10% by volume and are preferably present in a quantity ranging from 1-5% with respect to the total volume of gases present in the mixture.

The mixture of gases used for producing the microbubbles is apyrogenic and sterilized before use or packaging, for example by exposure to gamma rays or to ethylene oxide or other known sterilization forms.

In diagnostic use, the composition based on a mixture of O₂ and CO₂ according to the invention is systemically administered as such or combined with foaming carrying means. The administration is typically effected by intravenous injection.

Foaming carrying means which can be used are physiologically acceptable means which are appropriate contrast means for echography, once introduced into the circulatory stream. A typical carrying means comprises albumin but also polysorbate, in its various chemical forms.

For the specific uses of the invention, the composition is conveniently packaged and preserved inside a suitable container, typically consisting of a glass bottle for medical use.

The characteristics and advantages of the use of a mixture of oxygen and carbon dioxide, according to the present invention will appear more evident from the following illustrative and non-limiting description, referring to the enclosed schematic drawings, in which:

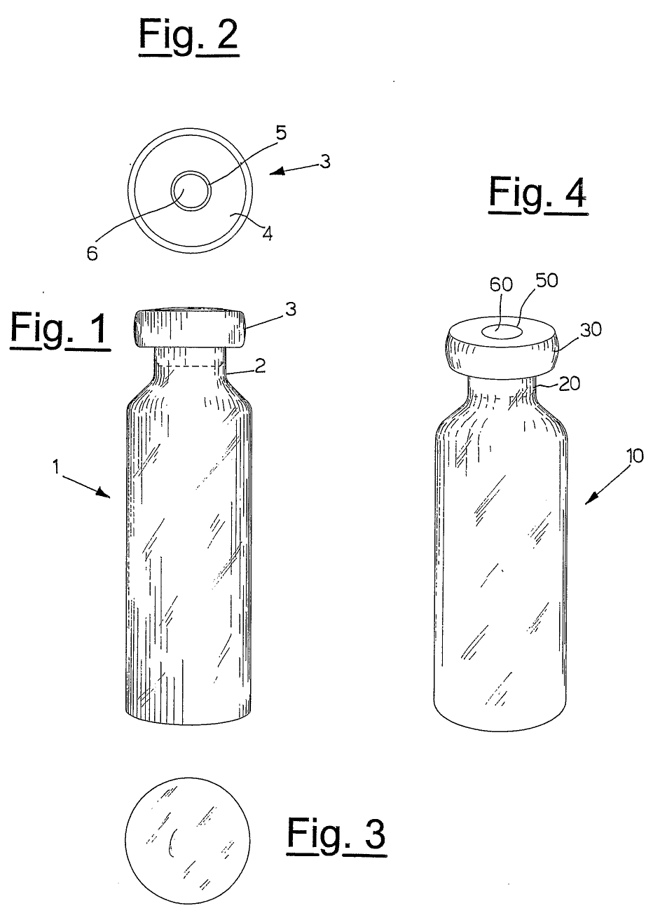

FIG. 1 shows a raised side view of a container for a composition of sterile gases of the invention;

FIG. 2 is a plan view from above of a container for a composition of sterile gases of the invention;

FIG. 3 illustrates a plan view from below of the container of the previous figures and

FIG. 4 shows a perspective of the container of the previous figures.

With reference to FIG. 1, this shows a container 1 for a composition of sterile gases of the invention, typically consisting of a glass bottle with a constriction or neck 2 on whose upper end a ring 3 typically made of metal, is attached. As can be observed from FIG. 2, said ring 3 has a covering portion 4 having a central hole 5. The metal ring 3 constrains a washer 6 to the upper end of the neck 2 of the bottle creating a tight seal and preventing the mixture of oxygen and gas contained in the container 1 from leaking.

FIG. 3 indicates that the lower end of the container 1 is closed and is made of the same material as the container. The washer is advantageously capable of offering a seal at an internal pressure ranging from 1.1 to 5 atmospheres. For this purpose, the ring 30 can be made of aluminum with a typical thickness varying from 0.1 to 2 mm. The bottle conveniently has a capacity varying from 5-10 cc approximately.

With reference to FIG. 4, in order to administer the composition of the invention to a patient for diagnostic purposes, it is sufficient to remove, for example with a syringe, a suitable quantity of mixture of O₂ and CO₂ by perforating the washer 60 of the container 10 with a needle and subsequently administering the gas mix, intravenously.

Analogously, it is possible to produce microbubbles suitable for therapeutic purposes by removing the gas mix from the bottle 10 with a syringe and extemporaneously preparing a sclerosing foam according to one of the techniques known to experts in the field which envisage the turbulent mixing of gas and a liquid.

According to an alternative embodiment, the bottle 10 is equipped with a filling and removal system of a fluid provided with valve means. More specifically, these valve means comprise a mobile shutting element between a position which allows the extraction or release of the fluid and a closed position. For this purpose, the perforating element has specific elastic means suitable for keeping the perforating element itself in a closed position.

It is possible for example to produce a sclerosing foam by resorting to: a) the method described by Tessari in US patent application 2002/0077589 A1 to whose text complete reference is made, b) the method described by A. L. Cabrera Gamido in Spanish patent application Nr. P 200301995 or alternatively, c) with the device for the production of a foam for diagnostic or therapeutic use described by Attilio Cavezzi and Lorenzo Tessari in Italian patent application MI 2003 #1204, incorporated herein as reference.

Once the sclerosing foam obtained has been injected into the vessel to be treated, thanks to the presence of oxygen, it proves to be capable of persisting for a sufficient time for allowing the action of the sclerosing agent on the innermost walls of the vessels. Furthermore, the corresponding presence of CO₂, which tends to permeate blood vessels more rapidly, allows a uniform distribution of the foam on the inner surfaces of the vessels.

The packaging in bottles having varying dimensions of gas mixtures for the uses of the invention enables the preparation procedure of microbubbles/foams for sclerosing therapy to be further standardized and simplified.

According to a preferred embodiment, in order to form a foam with microbubbles, the mixture containing O₂/CO₂ is mixed with a liquid or mixture of foaming liquids suitable for therapeutic or diagnostic use. The neo-formed foam is revealed by echographic/ultrasoundgraphic means currently available on the market.

In particular, the formation of foam is obtained by mixing, under turbulence conditions, a physiologically acceptable foaming liquid with the gaseous mixture based on CO₂/O₂ of the invention.

For the formation of a foam, a turbulence is required which can be created, for example, by passing the liquid and the gas mixture through a constriction/passage with a brusque enlargement. Optionally, it is possible to add a foaming substance such as a surface-active agent in order to improve the dispersion of gas bubbles.

When the method according to Tessari described for example in GB patent application 2 369 996 A is used, it is the passage of the sclerosing-gas liquid mixture inside a three-way tap which creates the turbulence and final foam. When, on the other hand, the method described by Antonio Cabrera Gamido, for example in patent Nr. P 200301995 is used, the disposable syringe contains the suitable quantity of sclerosing liquid and gas soluble in the blood at atmospheric pressure; the syringe is then shaken. The shaking of the syringe and its contents causes a dispersion with the liquid as a continuous system and the gas as a discontinuous system i.e. microbubbles.

The foaming liquid is conveniently a surface-active sclerosing drug for therapeutic use capable of forming a foam and sclerosing venous vessels, when injected into the lumen of the vessel.

Examples of suitable sclerosing substances comprise surface-active agents such as sodium tetradecyl sulfate, polydocanol, ethanolamine oleate, sodium morruate, or other sclerosing agents.

According to another embodiment, said liquid for diagnostic use is a biological foaming liquid for diagnostic use i.e. capable of forming a foam with a physiologically acceptable gas which can be revealed by means of an echographic/ultrasoundgraphic apparatus.

Biological foaming liquids comprise albumin, plasma, or inert and physiologically acceptable foaming substances.

According to another aspect of the present invention, a method is therefore provided for the production of a foam suitable for diagnostic or therapeutic application comprising the transfer of the mixture of CO₂ and O₂ of the invention and a pharmaceutically acceptable foaming liquid through one or more constrictions for mixing the gas and liquid under turbulence conditions and forming a foam and maintaining the foam under pressure until the moment of use.

The ratio of the gas mixture with respect to the liquid is advantageously pre-established so as to produce a foam in which the gaseous microbubbles are finely dispersed, creating a density conveniently ranging from 0.09 to 0.40 g/ml.

The ratio of the volume of the gas mixture with respect to the liquid preferably ranges from 2:1 to 10:1, more preferably from 3:1 to 7:1, and even more preferably is equal to 4:1 with the Tessari method described for example in GB patent application 2 369 996 A.

With the typical syringe shaker of the Antonio Cabrera method, the shaking time typically ranges from 30 seconds to 5 minutes. The liquid/gas ratio preferably ranges from 1 to 4. The dimension of the microbubbles is typically equal to about 50 microns, the density of the foam depends on the concentration of surface-active agent and varies from 0.2 to 4 g per cm³. In the case of the use of a sclerosing substance as a therapeutic foaming liquid, the concentration of the sclerosing agent is conveniently within the range of concentrations commonly used in sclerotherapy. Furthermore, in the case of a mixture with other types of drugs, for example local anesthetics, anti-inflammatory substances etc., foaming agents in themselves or made to be as such, the foam obtained with the gas mix herein disclosed, can be simply and extemporaneously produced with complete safety and sterility; the parenteral injection of said drugs in extravasal foam form, can in fact have advantages with respect to liquid or solid form such as a greater persistence in loco, a longer duration and, hypothetically, greater strength. The gas mixture of the invention is therefore useful in extravasal parenteral use due to the facility and innocuity of the reabsorption in the tissues of its two components (O₂ and CO₂).

The fact that the gas mixture of the invention allows a foam for therapeutic or diagnostic use to be extemporaneously produced and with greater standardization, is also particularly advantageous.

The possibility of using pre-established quantities of gas mix and therapeutic substance or diagnostic agent, adopting a foam production method which is also highly standardized, provides doctors with a procedure which is simple to put into practice and which also has a high safety profile.

In particular, a foam rich in microbubbles can be formed by sending a pre-established gas/liquid foaming mixture, for a preselected number of times inside the mixing device, for example a syringe which allows varying pressure values to be exerted on the gas/liquid mixture, by means of piston-like operation.

The following examples are provided for purely illustrative purposes of the present invention and should in no way be considered as limiting its protection scope as defined in the enclosed claims.

### EXAMPLE 1

A mixture containing CO₂ 60% by volume and O₂ 40% by volume is packaged in a 5 cc bottle at a pressure of 3 atmospheres. Once the gas mixture has been sterilized by means of gamma rays, it is ready for the extemporaneous formation of microbubbles.

### EXAMPLE 2

Microbubbles were produced, having a diameter of 6 microns by removing with a 5 cc syringe, a mixture of 65% of CO₂, 38% of O₂ and 2% of ozone contained in a sterile bottle. The gas mixture was mixed with sodium tetradecyl sulfate and/or polydocanol according to the method described by Tessari or Cabrera with the formation of a foam consisting of microbubbles which were injected into a vein to be sclerosed.

1. Use of a composition of sterile gases, soluble at atmospheric pressure and physiologically acceptable for the production of microbubbles as contrast means for ultrasounds, wherein said composition comprises oxygen and carbon dioxide and optionally at least another gas in a quantity not higher than 10% by volume with respect to the total volume of said mixture.

2. Use of a composition of sterile gases, soluble at atmospheric pressure and physiologically acceptable for the production of microbubbles for sclerotherapy, wherein said composition comprises oxygen and carbon dioxide and optionally at least another gas in a quantity not higher than 10% by volume with respect to the total volume of said mixture.

3. Use of a composition of sterile gases, soluble at atmospheric pressure and physiologically acceptable for the production of microbubbles for carrying at least one drug in the form of a foam for parenteral use, wherein said composition comprises oxygen and carbon dioxide and optionally at least another gas in a quantity not higher than 10% by volume with respect to the total volume of said mixture.

4. Use according to ante of the claim 1, wherein said mixture comprises CO₂ in a quantity ranging from 50 to 95% by volume with respect to the total volume of gas in the mixture.

5. Use according to claim 4, wherein the CO₂ is present in a quantity ranging from 60 to 80% by volume with respect to the total volume of gas in the mixture.

6. Use according to claim 1, wherein said other gas is selected from ozone, air, nitrogen, and mixtures thereof.

7. Use according to claim 1, wherein said gas mixture essentially consists of O₂ and CO₂.

8. Use according to claim 1, wherein the microbubbles produced have a diameter ranging from 10 to 50 microns.

9. Use according to claim 1, wherein said composition of sterile gases is packaged in a sterile disposable container.

10. Use according to claim 1, wherein said microbubbles are produced by mixing said composition of sterile gases, under turbulence, with a physiologically acceptable foaming liquid.

11. Use according to claim 10, wherein said foaming liquid is selected from albumin, plasma, polysorbates and mixtures thereof.

12. Use according to claim 1, wherein said microbubbles are produced by mixing said composition of sterile gases, under turbulence, with a sclerosing substance.

13. Use according to claim 12, wherein said foaming sclerosing substance is a sclerosing surface-active agent, or a sclerosing drug transferred into a foam by means of foaming additives.

14. Use according to claim 12, wherein said sclerosing substance is selected from sodium tetradecyl sulfate, polydocanol, ethanolamine oleate, sodium morruate, polydocanol and mixtures thereof.
